(19) United States
(12) Patent Application Publication
(10) Pub. No.: US 2021/0072257 A1
(43) Pub. Date: Mar. 11, 2021

(54) METHODS OF DIAGNOSING LYME DISEASE

(71) Applicant: MICROBPLEX, INC., Atlanta, GA (US)

(72) Inventors: Frances Eun-Hyung Lee, Atlanta, GA (US); John L. Daiss, Rochester, NY (US); Natalie Haddad, Atlanta, GA (US)

(21) Appl. No.: 17/015,913

(22) Filed: Sep. 9, 2020

Related U.S. Application Data
(60) Provisional application No. 62/897,839, filed on Sep. 9, 2019.

(51) Int. Cl. G01N 33/68

(52) U.S. Cl. CPC G01N 33/6854; G01N 2800/52; G01N 2800/26; G01N 2333/20

(57) ABSTRACT

Disclosed are methods for diagnosing Lyme disease and distinguishing a new infection from a persistent infection of Borrelia burgdorferi in a subject.

### CROSS-REFERENCE TO RELATED APPLICATIONS

This application claims the benefit of U.S. Provisional Application No. 62/897,839, filed Sep. 9, 2019, which is herein incorporated by reference in its entirety.

### STATEMENT REGARDING FEDERALLY SPONSORED RESEARCH OR DEVELOPMENT

This invention was made with Government Support under Grant No. R43AI134543 awarded by the National Institutes of Health. The Government has certain rights in the invention.

### BACKGROUND

Lyme disease in the United States is caused by Borrelia burgdorferi and Borrelia mayonii. Lyme disease is the most commonly reported arthropod-borne infection in the US with recent CDC estimates eclipsing 300,000 new cases in 2013. In addition to growing in frequency, the infections have a complex and increasingly severe course. Beginning with mild flu-like symptoms and frequently a signature bull's-eye rash, erythema migrans, Lyme disease can progress to severe articular, neurological and cardiac symptoms, most of which are preventable with early antibiotic therapy.

Lab tests to identify antibodies to the bacteria can help confirm or rule out the diagnosis. These tests are most reliable a few weeks after an infection, after anti-bacteria antibodies developed in infected humans. These tests include, 1) Enzyme-linked immunosorbent assay (ELISA) test, the test used most often to detect Lyme disease and 2) Western Blot test. ELISA detects antibodies to Borrelia burgdorferi. However, because it can sometimes provide false-positive results, it is not used as the sole basis for diagnosis. In addition, this test may not be positive during the early stage of Lyme disease, but the rash is distinctive enough to make the diagnosis without further testing in people who live in areas infested with ticks that transmit Lyme disease. If the ELISA test is positive, this test is usually done to confirm the diagnosis. In this two-step approach, the Western blot detects antibodies to several proteins of Borrelia burgdorferi.

However, leading investigators have identified two major shortcomings to the current serology-based methods for the definitive diagnosis of Early Localized Lyme disease. First, the clinical sensitivity in the first four weeks is poor, under 50% at the time of symptom onset, so many patients remain undiagnosed or unconfirmed until the disease has had time to progress. Second, serum antibody levels remain elevated long after the infection has been resolved making the monitoring of therapeutic success or diagnosis of re-infection virtually impossible. What is needed are new rapid highly specific and sensitive methods that can reliably detect borrelia infection without the limitations of current test.

### SUMMARY

Disclosed herein are methods for diagnosing, monitoring, and treating Lyme disease in a patient.

The details of one or more embodiments of the invention are set forth in the accompanying drawings and the description below. Other features, objects, and advantages of the invention will be apparent from the description and drawings, and from the claims.

Disclosed herein are methods of distinguishing a new Borrelia burgdorferi infection from a prior Borrelia burgdorferi infection in a subject (for example a human subject), said method comprising: obtaining a whole blood or a PBMC sample from the subject; separating newly proliferated antibody secreting cells (ASC) from the whole blood or the PBMC sample (including, but not limited to plasma cells, plasmablasts, or activated B cells); generating MENSA by incubating the ASC in a media for supporting antibody production from the ASC; and measuring levels of antibodies specific for antigens of Borrelia burgdorferi (including, but not limited to, whole protein and peptide antigens BBK32, BmpA, DbpA, Lal, OppA2, OspA, RevA, C6(N), C6(C), pepC10(N), and pepC10(C) or any combination as well as cross reactive Borrelia garinii variants of the same) in the MENSA; wherein the levels of the antibodies specific for the antigens of Borrelia burgdorferi higher than a control indicates a new Borrelia burgdorferi infection in the subject.

Disclosed herein are methods of distinguishing a new Borrelia burgdorferi infection from a prior Borrelia burgdorferi infection of any preceding aspect, wherein the subject has had prior Borrelia burgdorferi exposure, had a prior infection with Borrelia burgdorferi, and/or previously developed Lyme disease.

Also disclosed herein are methods of distinguishing a new Borrelia burgdorferi infection from a prior Borrelia burgdorferi infection of any preceding aspect, wherein the newly proliferated ASC are separated from the whole blood or the PBMC sample via Ficoll gradient separation or elutriation. In some embodiments, the newly proliferated ASC are separated from the whole blood or the PBMC sample via magnetic bead pull-down or fluorescence activated cell sorting. In some embodiments, magnetic bead pull-down or fluorescence activated cell sorting separates the newly proliferated ASC using antibodies specific for one or more cell surface markers comprising CD38, CD27, CD19, CD138, or IgD.

In some aspects, disclosed herein are methods of distinguishing a new Borrelia burgdorferi infection from a prior Borrelia burgdorferi infection of any preceding aspect, wherein the levels of the antibodies are determined by immunoassay comprising Enzyme-linked immunospot (ELISPOT), surface plasmon resonance, chemiluminescence, Enzyme-linked immunosorbent assay (ELISA), western blot, or a multiplex ELISA assay (including, but not limited to Luminex, Veriplex, LEGENDplex, Bio-Plex, Milliplex MAP, and FirePlex).

Also disclosed herein are methods of diagnosing the presence of a prior or a new Borrelia burgdorferi infection in a subject, said method comprising: obtaining a whole blood or a PBMC sample from the subject; separating newly proliferated antibody secreting cells (ASC) from the whole blood or the PBMC sample; generating MENSA by incubating the ASC in a media for supporting antibody production from the ASC; and measuring levels of antibodies specific for antigens of Borrelia burgdorferi in the MENSA; wherein the levels of the antibodies specific for the antigens of Borrelia burgdorferi higher than a control indicates a new Borrelia burgdorferi infection in the subject. In some aspect, the method further comprises treating a subject positively identified with a new or ongoing Borrelia burgdorferi infection with an antibiotic including, but not limited to doxycycline, amoxicillin, and/or cefuroxime.

In one aspect, disclosed herein are methods of treating, inhibiting, decreasing, reducing, and/or ameliorating a new or ongoing Borrelia burgdorferi infection in a subject said method comprising: a) obtaining a whole blood or a PBMC sample from the subject; b) separating newly proliferated antibody secreting cells (ASC) from the whole blood or the PBMC sample; c) generating MENSA by incubating the ASC in a media for supporting antibody production from the ASC; d) assaying levels of antibodies specific for antigens of Borrelia burgdorferi in the MENSA; wherein the levels of the antibodies specific for the antigens of Borrelia burgdorferi higher than a control indicates a new Borrelia burgdorferi infection in the subject; and e) administering a drug (such as, for example, an antibiotic including, but not limited to doxycycline, amoxicillin, and/or cefuroxime) to a subject that assays positive for new or ongoing Borrelia burgdorferi infection.

Also disclosed herein are methods of assessing the efficacy of a drug (such as, for example, an antibiotic including, but not limited to doxycycline, amoxicillin, and/or cefuroxime) in treating Lyme disease in a subject, said method comprising: obtaining a whole blood or a PBMC sample from the subject around day 3 to day 30 following treatment of the drug; separating an ASC from the whole blood or the PBMC sample; generating MENSA by incubating the ASC in a media for supporting antibody production from the ASC; measuring levels of antibodies specific for antigens of Borrelia burgdorferi in the MENSA; wherein the level of the antibodies specific for the antigens of Borrelia burgdorferi lower than a control indicates that the drug is effective.

### DESCRIPTION OF DRAWINGS

FIG. 1 shows the kinetics of Antibody Secreting Cells vs. Serum Titer Over Time. At the start of a new infection, newly stimulated ASCs begin circulating, reach a peak response within a few days, then decline in response to successful therapy (green line). The serum response typically lags by a few days to weeks and can last long after therapy is complete (yellow line). In cases where a patient was previously exposed to Lyme antigens prior to current infection, serum titers may start at a higher baseline (dashed yellow line), and a new infection is indistinguishable from an old one.

FIGS. 2A, 2B, 2C, and 2D show MicroB-plex Lyme Anti-C6 and Anti-pepC10 IgG+IgM immunoassays in MENSA and Serum of acute infected subjects. FIG. 2A shows that seven of the 10 confirmed Lyme disease patients were positive by the Anti-C6 MENSA assay (>22 MFI-B, red dashed line) within 25 days post symptom onset. FIG. 2B shows that five of the seven Anti-C6 MENSA positive patients had also seroconverted in Anti-C6 serum assays (>4565 MFI-B). Two MENSA negative subjects were seropositive possibly due to late sample collection (MENSA had already declined). FIG. 2C shows that in Anti-pepC10 MENSA assays, five of the ten confirmed Lyme subjects were identified (>20MFI-B). FIG. 2D shows that in Anti-pepC10 Serum assays, four of the five MENSA positive subjects were also detected (>2893MFI-B). One additional MENSA negative subject was detected by serum, again possibly due to late sample collection. Thus, a combined C6 and pepC10 MENSA assay identified 8/10 positive Lyme infections early in disease. MENSA assays can be improved by collecting samples earlier in the infection, prior to seroconversion. MFI-B: Median Fluorescence Intensity minus Background.

FIGS. 3A, 3B, 3C, 3D, 3E, and 3F show that kinetics of the MicroB-plex Lyme Anti-C6 IgG+IgM assay in MENSA and serum sample in three representative patients with Lyme infection. FIGS. 3A and 3B show an early positive Lyme C6 response in MENSA prior to a detectable serum response leads to an earlier diagnosis of Lyme disease. FIGS. 3C and 3D show a patient with acute Lyme shows a positive MENSA and serum response early in disease. After successful treatment, MENSA assay shows a rapid decline while serum antibody titers remain positive. FIGS. 3E and 3F show a patient with persistent symptoms including neurologic complications after antibiotic treatment. Although the serum response appears to be declining, it is clear from the MENSA that this patient is demonstrating an ongoing response. Graphs were created from representative patient data for each model. Black dotted line represents origin in MENSA graphs, red dashed line represents the cut-off value for positive in both MENSA and serum graphs: MENSA (22 MFI-B) and serum (4565 MFI-B) respectively. MFI-B: Median Fluorescence Intensity minus Background. Values represent Anti-C6 IgG+IgM.

FIG. 4 shows the serum response to 11 Borrelia burgdorferi antigens in two CDC standard sample sets. Seven proteins and two orientations of C6 and pepC10 peptides were coupled to Luminex beads and response was measured against standard serum sample collections from Center of Disease Control (CDC). Collection sets include serum from Stage 1 Early Lyme patients, Stage 2 Neurologic or Cardiac symptoms of advanced Lyme disease patients, and Stage 3 Lyme arthritis patients. Negative controls include healthy endemic and non-endemic subjects, as well as patients suffering fibromyalgia, rheumatoid arthritis, multiple sclerosis, mononucleosis, syphilis, severe periodontitis. Also tested was TcdB CROP, a non-Lyme marker specific for C. difficile which appears positive in a small fraction of non-Lyme controls.

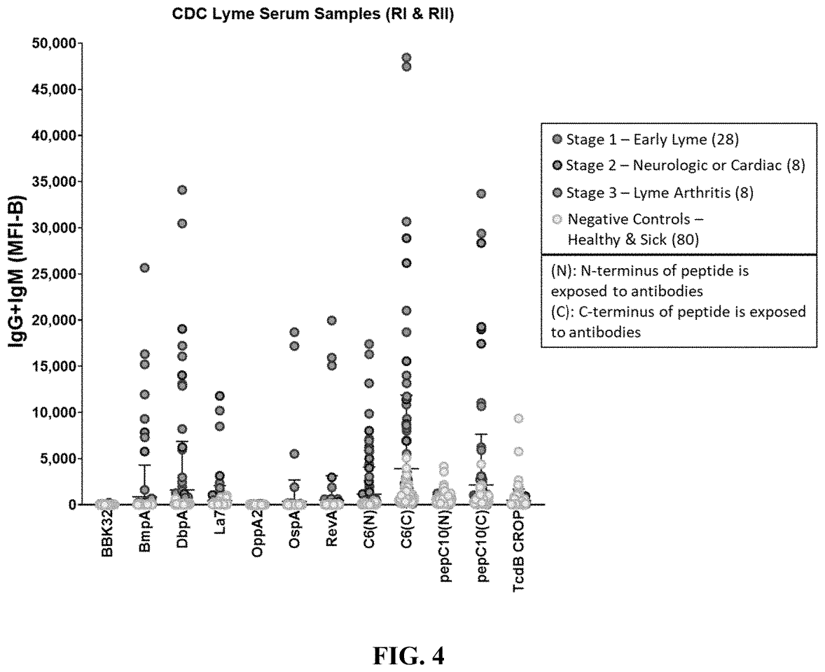

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

In the present invention, “specific” means a condition where one of the molecules involved in specific binding does not show any significant binding to molecules other than a single or a number of binding partner molecules. Furthermore. “specific” is also used when an antigen-binding domain is specific to a particular epitope among multiple epitopes contained in an antigen. When an epitope bound by an antigen-binding domain is contained in multiple different antigens, antigen-binding molecules containing the antigen-binding domain can bind to various antigens that have the epitope.

A “control” is an alternative subject or sample used in an experiment for comparison purposes. A control can be “positive” or “negative.”

A “variant” as used herein means a polypeptide or a polynucleotide encoding the polypeptide comprising one or more modifications such as substitutions, deletions and/or truncations of one or more specific amino acid residues or of one or more specific nucleotides or codons in the polypeptide or polynucleotide. The term “variant” as used herein is one that does not appear in a naturally occurring polynucleotide or polypeptide. A variant of a polynucleotide may be naturally occurring or synthetic, and may have 50%, 55%, 60%, 65%, 70%, 75%, 80%, 85%, 90%, 95%, 96%, 97%, 98%, or 99% identity with the wild type polynucleotide or a fragment thereof.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

“Antigen” means any native or foreign substance that is capable of eliciting an immune response. Preferably, the antigen will elicit an antibody, plasma cell, plasmablast, or B-cell response. Such antigens can include but are not limited to peptides and/or proteins from a subject, virus, bacteria, yeast, or parasite, including but not limited to toxins. Antigens can also include vaccines (e.g., peptides, proteins, killed pathogens, or attenuated pathogens administered in a pharmaceutically acceptable carrier either prophylactically or therapeutically), bio-warfare agents, and native peptides, polypeptides, and proteins. Antigen of the present invention are the antigens of Borrelia burgdorferi.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The fragments, whether attached to other sequences or not, include insertions, deletions, substitutions, or other selected modifications of particular regions or specific amino acids residues, provided the activity of the fragment is not significantly altered or impaired compared to the nonmodified antigen or the fragment thereof. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

Generally, either peripheral blood lymphocytes (“PBLs”) also referred to as peripheral blood mononuclear cells (PBMC) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high-level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against an antigen. Preferably, the binding specificity of monoclonal antibodies produced by the hybridoma cells is determined by immunoprecipitation or by an in vitro binding assay, such as radioimmunoassay (RIA) or enzyme-linked immunosorbent assay (ELISA). Such techniques and assays are known in the art and are described further in the Examples below or in Harlow and Lane Antibodies, A Laboratory Manual Cold Spring Harbor Publications, New York, (1988).

“Efficacy”, “efficacious”, “effective” or “sufficiency” means the ability to function as intended. For example, an “efficacious” immune response is a response that is able to afford the subject an acceptable degree of immune protection from the immunizing antigen. Thus, the present methods disclose methods of assessing the ability of an immune response to provide immune protection against future antigenic encounter. Traditionally, such methods involve antigenic challenge. It is understood that the present methods provide an alternative means to achieve the goal of antigenic challenge and can be used separately or in conjunction with a challenge to determine efficacy or sufficiency.

By “effective amount” is meant a therapeutic amount needed to achieve the desired result or results, e.g., establishing an immune response that can confer immunological protection to the subject. It is understood that immunological protection includes, but is not limited to, prevention of subsequent infections; reduction of the effects or symptoms of subsequent infections or conditions; reduction in the duration of the infection or condition; lessening of severity of a disease or condition; or reduced antigenic load relative to non-treated controls.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “treat,” “treating,” “treatment,” and grammatical variations thereof as used herein refer to the medical management of a patient with the intent to cure, ameliorate, stabilize, or prevent a disease, pathological condition, or disorder. These terms include active treatment, that is, treatment directed specifically toward the improvement of a disease, pathological condition, or disorder, and also includes causal treatment, that is, treatment directed toward removal of the cause of the associated disease, pathological condition, or disorder. Additionally, treatment includes partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, palliatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller gene expression, protein expression, amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

The terms “prevent,” “preventing,” “prevention,” and grammatical variations thereof as used herein, refer to a method of partially or completely delaying or precluding the onset or recurrence of a disease and/or one or more of its attendant symptoms or barring a subject from acquiring or reacquiring a disease or reducing a subject's risk of acquiring or reacquiring a disease or one or more of its attendant symptoms.

A “control” is an alternative subject or sample used in an experiment for comparison purposes. A control can be “positive” or “negative.”

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Compositions and Methods

Leading investigators have identified three major shortcomings to the current serology-based methods for the definitive diagnosis of Early Localized Lyme disease. First, the clinical sensitivity in the first four weeks is poor, under 50% at the time of symptom onset, so many patients remain undiagnosed or unconfirmed until the disease has had time to progress. Second, serum antibody levels remain elevated long after the infection has been resolved making the monitoring of therapeutic success or diagnosis of re-infection virtually impossible. Third, about 40% of confirmed Lyme patients never seroconvert using current serology testing. Accordingly, disclosed herein are methods of distinguishing a new Borrelia burgdorferi infection from a prior Borrelia burgdorferi infection in a subject.

In one aspect, the disclosed method uses Media containing ASC-Elaborated Newly synthesized Antibodies (MENSA) as the marker for recent exposure. MENSA comprises “newly synthesized antibodies” directly elaborated from specialized cells during acute illness or following vaccination. MENSA differs from serum in that the antibodies present in serum are from long-lived plasma cells in the bone marrow and not newly synthesized antibodies. MENSA also differs from media containing PBMC in that there are no memory B cells present and the ASC therein are secreting new synthesized antibody specific for an ongoing antigenic insult. Thus, put another way, disclosed herein is media substantially free of pre-existing antibody, but comprising newly synthesized antibodies from recently proliferating ASC in the blood. It is understood and herein contemplated that “media substantially free of pre-existing antibody,” refers to media where the amount of pre-existing antibody in the media is reduced at least 10², 10³, 10⁴, 10⁵, 10⁶, 10⁷ relative to the biological sample (e.g., blood, plasma, or PBMC) from which the newly proliferated ASC are separated. Alternatively, the “media substantially free of pre-existing antibody,” can refer to media where newly synthesized antibodies are at least 10², 10³, 10⁴, 10⁵, 10⁶, 10⁷ or more, more abundant than any pre-existing antibody.

MENSA

Following antigen exposure from vaccination or infection, naïve and/or memory B cells proliferate and differentiate into ASCs in lymph nodes. These ASCs are thought to be generated in activated lymph nodes, a compartment that is difficult to sample. These recently blasted ASCs leave the lymph node and burst into the circulation as they migrate to other tissue sites such as the bone marrow, spleen, or sites of inflammation presumably reflecting the migration of these effector cells to survival niches in the bone marrow and spleen. These highly informative cells are readily detectable from as little as 1-5 cc of blood and in the absence of measurable bystander effect on unrelated antigenic specificities. As opposed to historic serum antibodies produced by resident bone marrow plasma cells, newly synthesized antibodies reflect an ongoing immune response as they are secreted from newly formed antibody secreting cells (ASC) that burst dramatically in the peripheral blood just a few days after acute infections and can persist in the circulation for several weeks.

MENSA can function as a new serologic surrogate with similarly high specificities of antibodies, but with one major advantage: the pathogen-specific ASCs require only a single time point and can identify patients during the acute illness. Since the “historic or old” antibodies found in plasma are removed and only the newly synthesized antibodies measured from newly proliferated ASC, only antibodies or immune reactions to the current illness are measured. Another advantage of measuring MENSA over insensitive low affinity IgM measurements is reliability. MENSA can detect all antibody isotypes such as high affinity IgG or IgA as well as low affinity IgM antibodies which can increase its reliability. Herein is shown that the following characteristics of MENSA as measured by ASC ELISPOT assays: (1) high pathogen-specificity with no bystander effect (2) specificity only during acute illness and not during asymptomatic periods 1 (3) detection at the time of initial clinical presentation.

Using research ASC ELISPOT, it was shown that ASC in MENSA secrete antibodies with high specificity for the offending antigen; can be detected early after infection; and, in contrast to microbe isolation test (such as PCR), can be detected for significantly longer periods of time. In fact, MENSA is the first immune biomarker to reliably produce results with antibody specificity at the time of acute illness. Also using ASC ELISPOT assays, it was shown herein that the novel analyte provides sensitivity and specificity of 93% and 100% in adult patients with acute respiratory viral infection (i.e. Respiratory Syncytial Virus, RSV). The data also demonstrate the very high specificity of this analyte for the diagnosis of influenza infections. Current research grade ASC ELISPOT assays show that measurement of newly synthesized antibodies represents a powerful and innovative class of diagnostic assays. This design validates this novel diagnostic approach and demonstrates easy adaptability to two different categories of bioterrorism agents, a live viral pathogen and a toxin.

To isolate the MENSA analyte, MENSA is separated from pre-existing antibody and other circulating plasma cell and B-cell populations. A major candidate to improve the separation step is to capture the ASCs using magnetic beads bearing antibodies specific for cell surface markers unique to ASCs, specifically CD19 or CD38. Whole PBMCs or isolation of CD19+PBMCs were less efficient than density purified CD38+ enriched selections for both total IgG and influenza-specific ASC frequencies per mL of blood. This result is not surprising since CD19 expression can be slightly lower on ASC than naïve and memory B cells even though circulating ASCs have both CD19 and CD38 cell surface expression. Therefore, a custom blended panel of bead marker sets can be used which includes CD19 and CD38 and others to optimally isolate the circulating ASC fraction from whole blood. These commercially available bead isolation steps typically require magnetic bead-laden ASCs that are retained by a magnet while the remainder of the blood cells and plasma are washed away. Thus, in one aspect disclosed herein are methods of isolating MENSA comprising obtaining whole blood, plasma, or PBMC from a subject, separating the newly proliferated ASC from the whole blood, plasma, or PBMC, washing the newly proliferated ASC, and incubating the newly proliferated ASC in a media that supports maintenance of ASC and antibody secretion.

As used throughout this application, the term “washing” refers to a process of removing contaminants such as plasma, PBMC, whole blood, and pre-existing antibodies from population of cells of interest, such as, for example newly proliferated ASC. It is understood and herein contemplated that washing comprises the administration of an excess volume of washing solution to dilute any contaminants. It is understood that washing can comprise a means for separating the newly proliferated ASC from the excess washing solution such as, for example, centrifugation (also referred to as spinning). The washing solution can then be discarded, and washed cells re-suspended in a suitable media. In one aspect the wash solution can comprise any media suitable for said purpose including but not limited saline, buffered saline, and tissue culture media such as, for example MEM, DMEM, RPMI, Media 199, Opti-MEM, F10, Ham's F12, IMDM, each with or without serum, such as, Fetal Calf serum or Fetal Bovine serum. It is further understood that a rinse and spin wash cycle can be performed more than one time each time decreasing the contaminants and increasing the purity of the newly proliferated ASC. For example, the rinse and centrifugation cycle can be performed one, two, three, four, five, six, seven, eight, nine, ten, or more times. It is further understood that through washing, the purity of the sample can comprise at least a two, three, four, five, six, seven, eight, nine, ten, 15, 20, 25, 30, 40, 50, 60, 70, 80, 90, 100, 150, 200, 250, 300, 400, 500, 600, 700, 800, 900, 1000, 2000, 3000, 4000, 5000, 10³, 2×10³, 3×10³, 4×10³, 5×10³, 6×10³, 7×10³, 8×10³, 9×10³, 10⁴, 10⁵, 10⁶, 10⁷-fold reduction in plasma, PBMC, pre-existing ASC, and/or pre-existing antibodies relative to whole blood, plasma, pre-existing antibody, or PBMC. Accordingly, in one aspect, disclosed herein are method of isolating MENSA comprising obtaining whole blood, plasma, or PBMC from a subject, separating the newly proliferated ASC from the whole blood, plasma, or PBMC, washing the newly proliferated ASC, and incubating the newly proliferated ASC in a media that supports maintenance of ASC and antibody secretion, wherein the washed ASC comprise at least a two, three, four, five, six, seven, eight, nine, ten, 15, 20, 25, 30, 40, 50, 60, 70, 80, 90, 100, 150, 200, 250, 300, 400, 500, 600, 700, 800, 900, 1000, 2000, 3000, 4000, 5000, 10³, 2×10³, 3×10³, 4×10³, 5×10³, 6×10³, 7×10³, 8×10³, 9×10³, 10⁴, 10⁵, 10⁶, 10⁷-fold reduction in plasma, PBMC, pre-existing ASC, and/or pre-existing antibodies relative to whole blood, plasma, or PBMC.

It is understood and herein contemplated that there are many mechanisms that can be used to separate ASC from whole blood, plasma, or PBMC. For example, ASC can be separated using Ficoll gradients, elutriation, cell sorting methods such as magnetic bead sorting or fluorescence activated cell sorting (FACS). While careful multicolor flow cytometry can identify several distinct subsets of circulating ASC populations, the majority of these cells can be captured within the CD19+, CD27hi, CD38hi population containing >90% of recently proliferated cells as indicated by the almost universal expression of the nuclear proliferation antigen Ki67. Accordingly, where ASC are sorted based using magnetic beads or fluorescence, the sort is based on the presence and/or absence of one or more surface markers to which a tagged antibody can be bound. Examples of cell surface markers for separating newly formed ASC include but are not limited to CD38, CD27, CD19, CD138, and IgD. Thus, for example, in one aspect disclosed herein are methods of isolating MENSA comprising obtaining whole blood, plasma, or PBMC from a subject, separating the newly proliferated ASC from the whole blood, plasma, or PBMC, using magnetic bead separation comprising anti-CD38 beads, and incubating the newly proliferated ASC in a media that supports maintenance of ASC and antibody secretion.

In order for MENSA to be useful for detection of antigenic exposure or for diagnosis, secreted antibodies that are newly synthesized in response to activation of ASC must be present and pre-existing antibodies should be absent. Thus, the media should comprise sugars and amino acids needed for protein synthesis, newly activated ASC, and at least a two, three, four, five, six, seven, eight, nine, or ten log reduction in plasma or serum for the subject. Additionally, MENSA may contain survival factors such as IL-2, IL-6, IL-15, IL-21, and IFN-α, APRIL, enhancers of antibody secretion such as IL-21, other non-antibody secreting cells such as T cells or macrophage, but not red blood cells. Also, depending on the ASC separation method employed, the MENSA may also contain magnetic beads or compounds needed for rapid separation of ASC from whole blood or plasma.

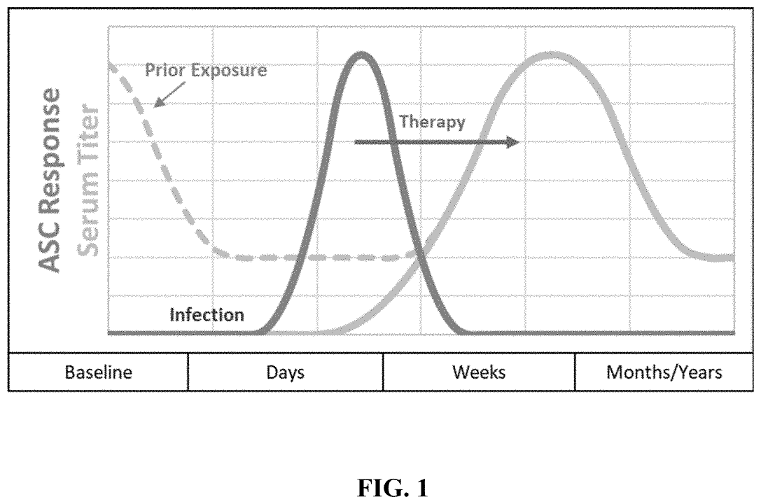

One of the primary drawbacks to using serum to measure Borrelia burgdorferi exposure is the kinetics of the serum response (FIG. 1). Serum antibody levels can take weeks to a few months to elevate to detectable levels and will persist at elevated levels never or slowly returning to background levels. This failure of serum levels to resolve or at a minimum resolve quickly conflates past infections with present infections and confounds diagnosis making it impossible to distinguish new from old infections. By contrast, MENSA antibody levels peak within a few days of exposure and decline to background levels, allowing for both fast detection/diagnosis and the ability to distinguish present infections from past infections. Additionally, while the background of MENSA is below detectable levels for anti-C6 and anti-pepC10 responses in controls, the base background level of serum antibodies in controls is above the detectable limits and has a standard deviation of means fluorescence intensity (MFI) well above 1000. In fact, compared to the Immunetics serum assay, the MicroB-plex C6 immunoassay using MENSA is has significantly better sensitivity. In fact, even using serum, the MicroB-plex C6 immunoassay was 30 times more sensitive than the Immunetics assay. In one aspect, disclosed herein are methods of measuring antigenic exposure (including a second or subsequent antigenic exposure) of a subject to Borrelia burgdorferi or diagnosing a subject with a new Borrelia burgdorferi infection comprising obtaining MENSA, antibodies, antibody secreting cells (ASC) or peripheral blood mononuclear cells (PBMC) from the subject between 3 and 45 days following antigenic exposure and measuring the number of ASC or antibodies from the isolated ASC, wherein the presence of ASC or antibodies specific for an antigen indicates recent antigenic exposure to said antigen thereby diagnosing the subject with a disease.

It is understood and herein contemplated that because the methods of measuring antigenic exposure or diagnosing a subject comprise obtaining MENSA; it is contemplated that the methods specifically contemplate the additional steps for obtaining MENSA into the disclosed diagnostic or antigen exposure methods. Thus, in one aspect, disclosed herein are methods of diagnosing or detecting the presence of a Borrelia burgdorferi infection (including, but not limited to detecting new infections, chronic infections, or sequelae from prior infections), diagnosing Lyme disease or detecting recent exposure to Borrelia burgdorferi in a subject comprising obtaining whole blood, plasma, or PBMC from the subject between 3 and 45 days following antigenic exposure; isolating newly formed ASC or antibody from the subject; washing the newly formed ASC, wherein the washed ASC comprise at least a two, three, four, five, six, seven, eight, nine, ten, 15, 20, 25, 30, 40, 50, 60, 70, 80, 90, 100, 150, 200, 250, 300, 400, 500, 600, 700, 800, 900, 1000, 2000, 3000, 4000, 5000, 10³, 2×10³, 3×10³, 4×10³, 5×10³, 6×10³, 7×10³, 8×10³, 9×10³, 10⁴, 10⁵, 10⁶, 10⁷-fold reduction in plasma, PBMC, pre-existing ASC, and/or pre-existing antibodies relative to whole blood, plasma, or PBMC; and measuring the number of ASC or antibodies from the isolated ASC specific for an antigen; wherein the presence of ASC or antibodies specific for the antigen indicates the subject as an ongoing infection with the source of the antigen. It is further contemplated herein that the disclosed methods can further comprise incubating the newly proliferated ASC in a media that supports maintenance of ASC and antibody secretion following the washing step and prior to measuring the number of ASC.

It is understood and herein contemplated that because MENSA antibodies specific for Borrelia burgdorferi antigens rise quickly and return to background levels, prior infections can be distinguished from new infections. Thus, in some aspects, disclosed herein is a method of distinguishing a new Borrelia burgdorferi infection from a prior Borrelia burgdorferi infection in a subject, said method comprising: obtaining a whole blood or a PBMC sample from the subject; separating newly proliferated antibody secreting cells (ASC) from the whole blood or the PBMC sample; generating MENSA by incubating the ASC in a media for supporting antibody production from the ASC; and measuring levels of antibodies specific for antigens of Borrelia burgdorferi in the MENSA; wherein the levels of the antibodies specific for the antigens of Borrelia burgdorferi higher than a control indicates a new Borrelia burgdorferi infection in the subject.

In some embodiments, the antigen of Borrelia burgdorferi comprises a whole protein such as, for example, BBK32 (Accession Number: AAL84596.1), BmpA (Accession Number: AAM89914.1), DbpA (Accession Number: ABD95800.1), Lal (Accession Number: AXK70357.1), OppA2 (Uniprot Accession Number: Q6RH12), OspA (Accession Number: CAA32579.1), RevA (Accession Number: WP 106019395.1), C6 peptide oriented with the N-terminus epitope exposed C6(N), C6 peptide oriented with the C-terminus epitope exposed (C), 10 amino acid peptide of OspC (pepC10) oriented with the N-terminus epitope exposed pepC10(N), and pepC10 oriented with the C-terminus epitope exposed pepC10(C) or any combination, a fragment or a variant thereof. In some embodiments, the antigen of Borrelia burgdorferi is a whole protein. In some embodiments, the antigen of Borrelia burgdorferi is a fragment thereof. In some embodiments, the antigen of Borrelia burgdorferi is a variant thereof. In some examples, the antigen of Borrelia burgdorferi are those described in U.S. Pat. Nos. 8,758,772B2, 7,887,815B2, 8,568,989B2, Amaboldi et al., Clin Vaccine Immunol. 2013 April; 20(4): 474-481. incorporated by reference herein in their entireties.

It is understood and herein contemplated that to avoid the results being confounded by pre-existing antibodies or circulating ASC, newly formed ASC should be separated in one aspect ASC isolated from the whole blood or plasma obtained from the subject. As noted herein, this separation can be achieved by Ficoll gradient centrifugation, elutriation and the like or by antibody means such as fluorescence activated cell sorting, magnetic bead sorting, and magnetic columns. Once separated, ASC can be washed one, two, three, four, five, six, seven, eight, nine, ten, or more times to remove residual contaminating PBMC, plasma, whole blood, or pre-existing antibodies. It is contemplated herein that the washed ASC comprise at least a two, three, four, five, six, seven, eight, nine, ten, 15, 20, 25, 30, 40, 50, 60, 70, 80, 90, 100, 150, 200, 250, 300, 400, 500, 600, 700, 800, 900, 1000, 2000, 3000, 4000, 5000, 10³, 2×10³, 3×10³, 4×10³, 5×10³, 6×10³, 7×10³, 8×10³, 9×10³, 10⁴, 10⁵, 10⁶, 10⁷-fold reduction in plasma, pre-existing ASC, PBMC, and/or pre-existing antibodies relative to whole blood, plasma, or PBMC. The newly formed ASC can then be cultured for 1, 2, 3, 4, 5, 6, 7, or 8 hours (i.e., from 1 to 2, 1 to 3, 1 to 4, 1 to 5, 1 to 6, 1 to 7, 1 to 8, 2 to 3, 2 to 4, 2 to 5, 2 to 6, 2 to 7, 2 to 8, 3 to 4, 3 to 5, 3 to 6, 3 to 7, 3 to 8, 4 to 5, 4 to 6, 4 to 7, 4 to 8, 5 to 6, 5 to 7, 5 to 8, 6 to 7, 6 to 8, or 7 to 8 hours) in a media conducive to production and expression of antibodies forming the analyte MENSA. The MENSA can comprise survival factors such as IL-2, IL-6, IL-15, IL-21, and IFN-α, APRIL, enhancers of antibody secretion such as IL-21, other non-antibody secreting cells such as T cells or macrophage, but not red blood cells. Also, depending on the ASC separation method employed, the MENSA may also contain magnetic beads or compounds needed for rapid separation of ASC from whole blood or plasma.

In one aspect, it is understood that detection can be accomplished by ELISA, chemiluminescence, ELISPOT, surface plasmon resonance, and hospital modular analyzers.

“Efficacy,” “efficacious,” or “sufficiency” means the ability to function as intended. For example, an “efficacious” immune response is a response that is able to afford the subject an acceptable degree of immune protection from the immunizing antigen. Thus, the present methods disclose methods of assessing the ability of an immune response to provide immune protection against future antigenic encounter. Traditionally, such methods involve antigenic challenge. It is understood that the present methods provide an alternative means to achieve the goal of antigenic challenge and can be used separately or in conjunction with a challenge to determine efficacy or sufficiency.

Throughout this application the term “sufficient immune response” is used to describe an immune response of a large enough magnitude to provide an acceptable immune protection to the subject against future pathogen encounter. It is understood that immune protection does not necessarily mean prevention of future antigenic encounter (e.g., infection), nor is it limited to a lack of any pathogenic symptoms. “Immune protection” means a prevention of the full onset of a pathogenic condition. Thus, in one embodiment, a “sufficient immune response” is a response that reduces the symptoms, magnitude, or duration of an infection or other disease condition when compared with an appropriate control. The control can be a subject that is exposed to an antigen before or without a sufficient immune response.

It is understood herein that an “immune response” refers to any inflammatory, humoral, or cell-mediated response that occurs for the purpose of eliminating an antigen. Such responses can include, but are not limited to, antibody production, cytokine secretion, complement activity, and cytolytic activity. In one embodiment, the immune response is an antibody response.

By “effective amount” is meant a therapeutic amount needed to achieve the desired result or results, e.g., establishing an immune response that can confer immunological protection to the subject. It is understood that immunological protection includes, but is not limited to, prevention of subsequent infections; reduction of the effects or symptoms of subsequent infections or conditions; reduction in the duration of the infection or condition; lessening of severity of a disease or condition; or reduced antigenic load relative to non-treated controls.

From an experimental standpoint, the assays and methods provided herein required optimization and adaptation of 3 steps of the current SOA ELISPOT: (1) separation of ASCs from plasma, (2) ASC secretion of newly expressed antibodies, and (3) detecting specificity of newly synthesized antibodies specific for Borrelia burgdorferi proteins.

Step 1: Typically, the ASCs can be separated from whole blood by Ficoll density centrifugation. Although relatively easy in a research laboratory, this process is time and labor intensive and not amenable to hundreds of samples that would be needed in a clinical laboratory during an outbreak; therefore, rapid and easy ASC isolation is contemplated. Circulating ASCs maintain CD19 cell surface expression and high levels of CD38 expression. These are lead candidate markers that can be used to isolate ASC. The data show that negative or positive magnetic ASC isolation of CD19+ and/or CD38+ cells from magnetic beads preserves spontaneous antibody production from the blood.

Step 2: Once newly blasted ASCs are separated from whole blood, they are washed in accordance with the disclosure herein. Washed ASC comprise at least a two, three, four, five, six, seven, eight, nine, ten, 15, 20, 25, 30, 40, 50, 60, 70, 80, 90, 100, 150, 200, 250, 300, 400, 500, 600, 700, 800, 900, 1000, 2000, 3000, 4000, 5000, 10³, 2×10³, 3×10³, 4×10³, 5×10³, 6×10³, 7×10³, 8×10³, 9×10³, 10⁴, 10⁵, 10⁶, 10⁷-fold reduction in plasma, PBMC, pre-existing ASC, and/or pre-existing antibodies relative to whole blood, plasma, or PBMC.

Step 3: Once newly blasted ASCs are separated from the serum antibodies, ASC are cultured for antibody secretion or elaboration. Circulating ASCs removed directly ex vivo secrete enough pathogen-specific antibodies within 1 hour as measured by ELISPOT immunoassays. Therefore, less than 1 hour is sufficient for the production of measurable antibodies. Shorter antibody elaboration times reduce antibody amounts. In another embodiment antibody secretion is enhanced from these cells with cytokines such as IL-2, IL-6, IL-15, IL-21, and IFN-α as well as other potential enhancers.

Step 4: The standard detection method by the ASC ELISPOT is highly sensitive with low background noise since it measures antibodies from a single cell. Unfortunately, ELISPOT assays can be time intensive and difficult to automate. Hence, measuring pathogen-specificity from total secreted antibodies from an entire population of isolated ASCs makes the process more streamlined. The data illustrate secretion rates from an individual circulating ASC at 10 pg per hour. However, higher secreted antibody concentrations can be achieved by increasing the blood sample volume (or more ASC) and/or by decreasing the elaboration volume or concentrating MENSA. The pathogen-specific antibodies are measured by conventional enzyme-linked methods which can detect in range of ng/mL and/or by chemiluminescence which has reliable detection limits of pg/mL (10⁻¹² M). Thus, these two methods were incorporated to validate MENSA fluid. There are two major advantages of MENSA. First, lower limits of microbe-specific antibodies in MENSA are well within the limits of detection of modular analyzers in most hospitals. The second major advantage of the MENSA fluid is the low background due to a single analyte (only the newly synthesized antibodies) captured in the media. This is unlike serum that contains numerous proteins including all types of antibodies. Because of its cleanliness, the MENSA matrix is amenable to label-free detection methods and many ultra-sensitive detector methods due to the high signal-to-noise ratio. Thus, in one aspect, detection can be accomplished by ELISA, chemiluminescence, ELISPOT, surface plasmon resonance, and hospital modular analyzers.

The methods disclosed herein comprise assessing the efficacy or sufficiency of an immune response to a selected antigen in a subject as well as diagnosing a disease or identifying antigen exposure in a subject. The disclosed methods utilize tissue samples from the subject to provide the basis for assessment. Such tissue samples can include, but are not limited to, blood (including peripheral blood and peripheral blood mononuclear cells), tissue biopsy samples (e.g., spleen, liver, bone marrow, thymus, lung, kidney, brain, salivary glands, skin, lymph nodes, and intestinal tract), and specimens acquired by pulmonary lavage (e.g., bronchoalveolar lavage (BAL)). Thus, it is understood that the tissue sample can be from both lymphoid and non-lymphoid tissue. Examples of non-lymphoid tissue include but are not limited to lung, liver, kidney, and gut. Lymphoid tissue includes both primary and secondary lymphoid organs such as the spleen, bone marrow, thymus, and lymph nodes. In one aspect the analyte under examination is MENSA.

In one aspect, the methods disclosed herein make diagnoses, measure exposure, or determine efficacy, effectiveness, or need for further treatment through the measure of the number or presence of antibody secreting cells (ASC) or antibodies. It is understood and herein contemplated that “antibody secreting cell” or “plasma cell” refers to any B lineage cell capable of secreting antibody including but not limited to plasmablasts, short-lived antibody secreting cells, long-lived plasma cell. It is further understood and specifically contemplated that the presence or number of such cells can be determined by any of the immunoassays disclosed herein, including but not limited to ELISPOT assay. It is further understood that where an ELISPOT assay is used to measure the presence or level of antibody secreting cells to a particular antigen, that ELISPOT assay can be antigen specific.

It is understood and herein contemplated that the disclosed diagnostic and/or detection methods and methods of distinguishing a new Borrelia burgdorferi infection from a prior Borrelia burgdorferi infection can be performed with MENSA, ASC, or antibodies obtained 3, 4, 5, 6, 7, 8, 9, 10, 11, 12, 13, 14, 15, 20, 25, 30, 35, 40, 45, 60, 75, 90, 120, or 150 days following antigenic exposure (including but not limited to infection and vaccination). In one aspect the PBMC are obtained between 3 and 45 days following antigenic exposure. In another aspect, the PBMC are obtained between 3 and 30 days following antigenic exposure. In another aspect, the PBMC are obtained between 3 and 20 days following antigenic exposure. In another aspect, the PBMC are obtained between 3 and 15 days following antigenic exposure. In another aspect, the PBMC are obtained between 3 and 12 days following antigenic exposure. In another aspect, the PBMC are obtained between 3 and 10 days following antigenic exposure. In another aspect, the PBMC are obtained between 5 and 10 days following antigenic exposure. In yet another aspect, the PBMC are obtained between 5 and 8 days following antigenic exposure.

In some embodiments, the newly proliferated ASC are separated from the whole blood or the PBMC sample via Ficoll gradient separation or elutriation. In some embodiments, the newly proliferated ASC are separated from the whole blood or the PBMC sample via magnetic bead pull-down or fluorescence activated cell sorting. In some embodiments, magnetic bead pull-down or fluorescence activated cell sorting separates the newly proliferated ASC using antibodies specific for one or more cell surface markers comprising CD38, CD27, CD19, CD138, or IgD.

In some embodiments, the ASC comprise plasma cells, plasmablasts, or activated B cells. In some embodiments, the antibodies are present in MENSA.

It is understood and herein contemplated, that the disclosed methods of distinguishing a new Borrelia burgdorferi infection from a prior Borrelia burgdorferi and methods of detecting or diagnosing the presence of a prior or a new Borrelia burgdorferi infection in a subject while utilizing MENSA, do not have to be performed to the exclusion of serum based detection. Thus, in one aspect, disclosed herein are methods of distinguishing a new Borrelia burgdorferi infection from a prior Borrelia burgdorferi and methods of detecting or diagnosing the presence of a prior or a new Borrelia burgdorferi infection in a subject further comprising measuring levels of antibodies specific for antigens of Borrelia burgdorferi in the serum.

In some embodiments, the subject is a human. In some embodiments, the human has or is suspected of having Borrelia burgdorferi infection. In some embodiments, the human has Borrelia burgdorferi infection. In some embodiments, the human has a new Borrelia burgdorferi infection. In some embodiments, the human has a prior Borrelia burgdorferi infection. In some embodiments, the human is suspected of having Borrelia burgdorferi infection.

It is understood that one goal of the disclosed detection or diagnostic methods and methods of distinguishing prior and present infections disclosed herein is to quickly and accurately diagnose a subject so treatment can be more rapidly be administered when appropriate. Accordingly, disclosed herein are methods of diagnosing or detecting the presence of a Borrelia burgdorferi infection (including, but not limited to detecting new infections, chronic infections, or sequelae from prior infections) wherein when a new Borrelia burgdorferi infection is detected, the method further comprises treating the Borrelia burgdorferi infection by administering to the subject a therapeutically effective amount of a drug (such as, for example, an antibiotic including, but not limited to doxycycline, amoxicillin, and/or cefuroxime) for treating Lyme disease. Thus, the disclosed detection or diagnostic methods can become treatment methods. Therefore, disclosed herein are methods of treating, inhibiting, decreasing, reducing, and/or ameliorating a new or ongoing Borrelia burgdorferi infection in a subject said method comprising a) obtaining a whole blood or a PBMC sample from the subject; b) separating newly proliferated antibody secreting cells (ASC) from the whole blood or the PBMC sample; c) generating MENSA by incubating the ASC in a media for supporting antibody production from the ASC; d) assaying levels of antibodies specific for antigens of Borrelia burgdorferi in the MENSA; wherein the levels of the antibodies specific for the antigens of Borrelia burgdorferi higher than a control indicates a new Borrelia burgdorferi infection in the subject; and e) administering a drug (such as, for example, an antibiotic including, but not limited to doxycycline, amoxicillin, and/or cefuroxime) to a subject that assays positive for new or ongoing Borrelia burgdorferi infection.

Immunoassays

As shown herein, ASC can be detected by ELISPOT, but the transient presence of the antigen-specific ASC could also be used by detection of antigen-specific antibody secreted by the cells by ELISA which may be easier for clinical diagnostic laboratories to perform. ASC may also be detected by an immuno-array or similar protein array or microarray. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known, and all are suitable for detecting the disclosed biomarkers. Examples of immunoassays are enzyme linked immunosorbent assays (ELISAs), enzyme linked immunospot assay (ELISPOT), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immune complexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label. See, for example, U.S. Pat. Nos. 3,817,837; 3,850,752; 3,939,350; 3,996,345; 4,277,437; 4,275,149 and 4,366,241, each of which is incorporated herein by reference in its entirety and specifically for teachings regarding immunodetection methods and labels.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorimetric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically, fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine 0; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; CyS™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (Di1C18(5)); DIDS; Dihydorhodamine 123 (DHR); Dil (Di1C18(3)); I Dinitrophenol; DiO (Di0C18(3)); DiR; DiR (Di1C18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow SGF; GeneBlazer; (CCF2); GFP (S65T); GFP red shifted (rsGFP); GFP wild type' non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin EBG; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the aspect include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avidin pair. In this mode, the detecting antibody or detecting molecule should include the other member of the pair.

Other modes of indirect labeling include the detection of primary immune complexes by a two-step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of measuring the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in a hemagglutination assay) or very small particles (as in a latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date back to the discovery of antibodies and none of them have an actual “label” (e.g. Ag-enz). Other kinds of immunoassays that are label-free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunological analysis.

The use of immunoassays to detect a specific protein can involve the separation of the proteins by electrophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally, the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10 MW for known samples and read off the log Mr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, NG, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each of which is herein incorporated by reference in its entirety for teachings regarding electrophoresis methods.

Laemmli, U.K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromogenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, that utilize enzyme-labeled secondary antibodies labeled with enzymes (e.g., alkaline phosphatase or horeradish peroxidase) that can activate chromogenic or chemiluminescent substrates. Other possibilities for probing include the use of fluorescent or radioisotope labels (e.g., fluorescein, ¹²⁵O. Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Ornstein L., Disc electrophoresis—I: Background and theory, Ann. NY Acad. Sci. 121:321-349 (1964), and Matsudiara, PT and DR Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, which is herein incorporated by reference in its entirety for teachings regarding gel shift methods.

Gel shift methods can include using, for example, colloidal forms of Coomassie blue stain (Imperial Chemicals Industries, Ltd) to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, Protein A-Sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunospot Assay (ELISPOT) is an immunoassay that can detect an antibody specific for a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. For descriptions of ELISA procedures, see Voller, A. et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, 1980; Butler, J. E., In: Structure of Antigens, Vol. 1 (Van Regenmortel, M., CRC Press, Boca Raton, 1992, pp. 209-259; Butler, J. E., In: van Oss, C. J. et al., (eds), Immunochemistry, Marcel Dekker, Inc., New York, 1994, pp. 759-803; Butler, J. E. (ed.), Immunochemistry of Solid-Phase Immunoassay, CRC Press, Boca Raton, 1991); Crowther, “ELISA: Theory and Practice,” In: Methods in Molecule Biology, Vol. 42, Humana Press; New Jersey, 1995; U.S. Pat. No. 4,376,110, each of which is incorporated herein by reference in its entirety and specifically for teachings regarding ELISA methods.

Variations of ELISA techniques are known to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISAs, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immune complexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immune complex (antigen/antibody) formation. Detection of the immune complex then requires a labeled secondary binding agent or a secondary binding agent in conjunction with a labeled third binding agent.

“Under conditions effective to allow immune complex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immune complexes between the test sample and the originally bound material, and subsequent washing, the occurrence of even minute amounts of immune complexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immune complex with a labeled antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialized chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include color coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately, and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilized on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Ariz.), rolling circle DNA amplification (Molecular Staging, New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staphylococcus aureus protein A (Affibody, Bromma, Sweden), ‘Trinectins’ based on fibronectins (Phylos, Lexington, Mass.) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, Colo.). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photo-crosslinking to ligand reduces the cross-reactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colors. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which can be used diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumor extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally, they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilized on a microarray. Large-scale ‘proteome chips’ promise to be very useful in identification of functional interactions, drug screening, etc. (Proteometrix, Branford, Conn.).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e., through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (1), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively. In some embodiments, the antibodies present in MENSA are IgGs. In some embodiments, the antibodies present in MENSA are IgMs.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain antigen binding activity are included within the meaning of the term “antibody or fragment thereof” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

In some embodiments, the levels of the antibodies are determined by immunoassay comprising Enzyme linked immunospot (ELISPOT), Enzyme-linked immunosorbent assay (ELISA), western blot, or a multiplex ELISA assay. In some embodiments, the multiplex ELISA assay is selected from the group consisting of Luminex, Veriplex, LEGENDplex, Bio-Plex, Milliplex MAP, and FirePlex.

In some aspects, disclosed herein is a method of diagnosing the presence of a prior or a new Borrelia burgdorferi infection in a subject, said method comprising: obtaining a whole blood or a PBMC sample from the subject; separating newly proliferated antibody secreting cells (ASC) from the whole blood or the PBMC sample; generating MENSA by incubating the ASC in a media for supporting antibody production from the ASC; and measuring levels of antibodies specific for antigens of Borrelia burgdorferi in the MENSA; wherein the levels of the antibodies specific for the antigens of Borrelia burgdorferi higher than a control indicates a new Borrelia burgdorferi infection in the subject.

In some aspects, disclosed herein is a method of assessing the efficacy of a drug in treating Lyme disease in a subject, said method comprising: obtaining a whole blood or a PBMC sample from the subject around day 3 to day 30 following treatment of the drug; separating an ASC from the whole blood or the PBMC sample; generating MENSA by incubating the ASC in a media for supporting antibody production from the ASC; measuring levels of antibodies specific for antigens of Borrelia burgdorferi in the MENSA; wherein the level of the antibodies specific for the antigens of Borrelia burgdorferi lower than a control indicates that the drug is effective.

In some embodiments, the whole blood or the PBMC sample is collected from the subject around day 3 to day 30 (e.g., around day 3, 4, 5, 6, 7, 8, 9, 10, 11, 12, 13, 14, 15, 16, 1, 18, 19, 20, 21, 22, 23, 24, 25, 26, 27, 28, 29, 30, 31, 32, 33, 34, or 35) following treatment of the drug.

In some embodiments, the drug comprises one or more antibiotics. In some embodiments, the one or more antibiotics are selected from the group consisting of doxycycline, amoxicillin, and cefuroxime.

In some embodiments, the one or more antibiotics are administered orally, intravenously, sublingually, topically (preferably ocularly, transdermally, nasally), or intraperitoneally. In some embodiments, the one or more antibiotics are administered orally. In some embodiments, the one or more antibiotics are administered intravenously.

Dosing frequency for the one or more antibiotics, includes, but is not limited to, at least about once every month, once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day. In some embodiments, the dosing frequency for the one or more antibiotics includes, but is not limited to, at least once a day, twice a day, three times a day, or four times a day. In some embodiments, the interval between each administration is less than about 48 hours, 36 hours, 24 hours, 22 hours, 20 hours, 18 hours, 16 hours, 14 hours, 12 hours, 10 hours, 9 hours, 8 hours, 7 hours, 6 hours, or 5 hours. In some embodiments, the interval between each administration is less than about 24 hours, 22 hours, 20 hours, 18 hours, 16 hours, 14 hours, 12 hours, 10 hours, 9 hours, 8 hours, 7 hours, 6 hours, or 5 hours. In some embodiments, the interval between each administration is constant. For example, the administration can be carried out daily, every two days, every three days, every four days, every five days, or weekly. Administration can also be continuous and adjusted to maintaining a level of the compound within any desired and specified range. The administration of the one or more antibiotics can be extended over an extended period of time, such as from about a month or shorter up to about three years or longer. For example, the dosing regimen can be extended over a period of any of about 2, 3, 4, 5, 6, 7, 8, 9, 10, 11, 12, 18, 24, 30, and 36 months. In some embodiments, there is no break in the dosing schedule. In some embodiments, the interval between a course of administration is no more than about a week

The efficacy and effectiveness of an antibiotic, for treating, inhibiting, or preventing a Borrelia burgdorferi infection can be assessed in various ways well known to the skilled practitioner. For instance, one of ordinary skill in the art will understand that an antibiotic is efficacious in treating or inhibiting a Borrelia burgdorferi infection in a subject by observing that the drug reduces bacterial load or prevents a further increase in Borrelia burgdorferi bacterial load. Borrelia burgdorferi bacterial loads can be measured by methods that are known in the art, for example, using polymerase chain reaction assays to detect the presence of Borrelia burgdorferi nucleic acid or by measuring the level of circulating anti-Borrelia burgdorferi antibody levels in the subject.

Because MENSA levels are more dynamic than serum levels which take longer to rise above background and longer to dissipate, it is understood and herein contemplated that the disclosed methods can also be used not only to assess the efficacy of a drug in treating Lyme disease, but also monitor the host response to treatment, with a decrease in MENSA levels following the administration of treatment indicating successful therapeutic intervention or host immune response. Thus, in one aspect, disclosed herein are methods of monitoring a subjects response to treatment for a Borrelia burgdorferi infection, said method comprising: obtaining a whole blood or a PBMC sample from the subject around day 3 to day 30 following treatment of the drug; separating an ASC from the whole blood or the PBMC sample; generating MENSA by incubating the ASC in a media for supporting antibody production from the ASC; and measuring levels of antibodies specific for antigens of Borrelia burgdorferi in the MENSA; wherein a decreased level of the antibodies specific for the antigens of Borrelia burgdorferi relative to a control indicates that the subject is responsive to the treatment. In some aspects the method can further comprise obtaining a serum sample and measuring the levels of antibodies specific for antigens of Borrelia burgdorferi in the serum.

### EXAMPLES

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. While the invention has been described with reference to particular embodiments and implementations, it will be understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention not be limited to the particular implementations disclosed herein, but that the invention will include all implementations falling within the scope of the appended claims.

### Example 1: MENSA as Measured by ASC ELISpots is an Excellent Immunodiagnostic Approach

Prior to investing in a rapid clinically-based assay, evidence that this novel body fluid is a useful diagnostic test was needed. Therefore, these results were confirmed using human clinical samples in a common respiratory infection, RSV disease, by developing research-based RSV-specific ASC ELISPOT assays. A critical aspect of this assay included separation of the ASC from the plasma that was teaming with historical antibodies. These cells were given time to secrete antibodies (which were highly pathogen-specific during infection) on an RSV antigen-bound membrane. Thus, only RSV-specific newly secreted antibodies were immediately captured and detected. The results showed that measurements of MENSA identified patients with >90% sensitivity and nearly 100% specificity who had PCR confirmed acute RSV infections.

This study was performed in both inpatients and outpatients or real-life scenarios at the time of initial clinical presentation. Thirty-nine patients with PCR confirmed RSV infections were enrolled, and this new diagnostic test identified 35/39 accurately at clinical presentation. In some patients, positive identification was made as early as the 2nd day of symptoms. This test provided results in real-time and not after the patient recovered which is often the case for most immune-based diagnostics. Unlike serum, MENSA by ASC ELISPOT assays appeared days earlier probably due to early appearance of the cells and the increased sensitivity 18. In conclusion, MENSA as a diagnostic is highly sensitive and specific as early as day 2 of illness and beyond.

### Example 2: MENSA is Very Specific after Recent Vaccination and Infection

Indirect evidence indicated that non-specific memory B cells differentiate during vaccination and form circulating ASC. However, the data shown herein shows no evidence of bystander ASC responses after vaccinations (with antigens such as tetanus toxoid, hepatitis B surface antigen, and human papilloma virus VLPs). Influenza vaccination stringently induces abundant circulating ASCs specific for influenza proteins but does not expand ASC reacting with other pathogens either by cross-reactivity or by non-specific polyclonal activation of memory B cells against unrelated antigens. Also, circulating ASC present in 28 healthy adults in the absence of immunization do not contain detectable responses against influenza or any other major pathogens. This exquisite specificity holds true during acute respiratory viral infections such as RSV and influenza virus whereby false positive are minimized. Thus, the nearly 100% specificity that was found. In conclusion, ASC responsible for MENSA represent excellent biomarkers for diagnosis of a microbial infection due to the combination of several salient properties. First, the abundance of circulating antigen-specific ASC cells increases greater than 500-fold in the peripheral blood after vaccination or infection.

Second which is of foremost importance, these cells secrete antibodies with amazing specificity for the microbe of recent exposure. Combined, the absence of baseline responses (ensuring very low experimental noise in the assay) and lack of bystander responses represent crucial elements for this diagnostic assay. Dual specificity means dual infections. A powerful illustration of the diagnostic potential of MENSA is provided by its ability to detect simultaneous infections with different organisms. MENSA specific for both an RSV and influenza on day 8 from one patient recruited with confirmed RSV infection by PCR 1. These findings suggested two possibilities: a unique massive bystander response or a simultaneous RSV and influenza virus infection. The latter was confirmed when repeat nasopharyngeal PCR tests revealed a co-infection with influenza virus confirming the ASC specificities. In conclusion, dual ASC specificities exemplify true co-infections.

### Example 3: Circulating ASCs are Special and Characteristically Different from Long-Lived Plasma Cells

During infection or vaccination, naïve or memory B cells (in different frequencies for primary versus recall responses), undergo massive proliferation and differentiation into ASCs in the draining lymph nodes then explode into the blood as they migrate to other tissue sites such as the bone marrow for permanent residence. The relationship between circulating ASCs and long-lived plasma cells is not well understood although many models have been proposed. Focus was not on the immune models but exploited the differences of these two cell types by their phenotype, kinetics, location, and secreted antibody specificity for the development of this new diagnostic test.

### Example 4: Circulating ASCs are Actively Proliferating in Response to Recent Antigenic Stimulation Whereas Long-Lived Plasma Cells are Resting Cells

Circulating ASCs represent the plasmablast population actively responding to an ongoing antigenic insult whereas resident bone marrow long-lived plasma cells are responsible for the historical antibodies found in serum. Studies demonstrate that, while careful multicolor flow cytometry can identify several distinct subsets of circulating ASC populations, the majority of these cells can be captured within the CD19+, CD27hi, CD38hi population containing >90% of recently proliferated cells as indicated by the almost universal expression of the nuclear proliferation antigen Ki67. In contrast, the majority of human bone marrow plasma cells are CD138+CD27hi CD38hi with less than 5% expressing Ki67 indicating many are non-dividing.

In the following experiments (unless specified), ASCs are separated from plasma using traditional Ficoll density centrifugation of peripheral blood mononuclear cells (PBMC) which contain the ASCs. MENSA is predicated on the principle of antigen specificity of contemporary antibodies newly generated in real time during acute infection. This is in contra-distinction to pre-existing serum antibodies which denote simply the presence of long-lived bone marrow plasma cells generated at the time of prior infections and which are therefore ineffectual if not misleading in the context of acute infection. Of note, this spontaneous antibody secretion assay does not measure resting memory B cells since memory B cells do not secrete antibodies spontaneously but require a prerequisite 4-6 days of in vitro proliferation and differentiation to secrete antibodies.

### Example 5: Detection of Borrelia burgdorferi

Early in an infection, pathogen-specific B lymphocytes are activated to form plasmablasts or antibody-secreting cells (ASCs) that enter and transiently circulate in blood. These circulating ASCs are present in the blood only during active infection and their numbers decline rapidly when the infection is resolved. By harvesting ASCs, and culturing them in a virgin cell culture medium, the ASCs create a novel matrix, MENSA (medium enriched for newly synthesized antibody). MENSA is an ideal analytical medium; it contains easily measurable quantities of newly synthesized, pathogen-specific antibodies and it has no antibodies from prior infections. ASCs emerge into the blood prior to seroconversion, so measurement of anti-Borrelia burgdorferi antibodies in MENSA facilitates earlier diagnosis of Lyme disease. Unlike serum antibodies, circulating ASCs disappear when the infection resolves, so levels of anti-Borrelia burgdorferi antibodies in MENSA can be used to track the success of antibiotic therapy. MENSA helps run early diagnosis and track therapeutic effect by following subjects with early Lyme disease through diagnosis and therapy over a period of months. This technology creates important new opportunities for diagnosis of Lyme disease and can be extended to other infectious diseases. As shown in FIG. 1, the comparative assay for detecting Lyme disease in patients using Mensa or Serum. Seven of the 10 confirmed Lyme disease patients were positive by the Anti-C6 MENSA assay (>22 MFI-B, red dashed line) within 25 days post symptom onset. However, FIG. 1B shows that only five of the seven Anti-C6 MENSA positive patients had also seroconverted in Anti-C6 serum assays (>4565 MFI-B) showing that MENSA is able to detect exposure earlier than serum-based tests. Two MENSA negative subjects were seropositive possibly due to late sample collection (MENSA had already declined). FIG. 1C shows that in Anti-pepC10 MENSA assays, five of the ten confirmed Lyme subjects were identified (>20MFI-B). FIG. 1D shows that in Anti-pepC10 Serum assays, four of the five MENSA positive subjects were also detected (>2893MFI-B). One additional MENSA negative subject was detected by serum, again possibly due to late sample collection. Thus, a combined C6 and pepC10 MENSA assay identified 8/10 positive Lyme infections early in disease. MENSA assays can be improved by collecting samples earlier in the infection, prior to seroconversion. MFI-B: Median Fluorescence Intensity minus Background.

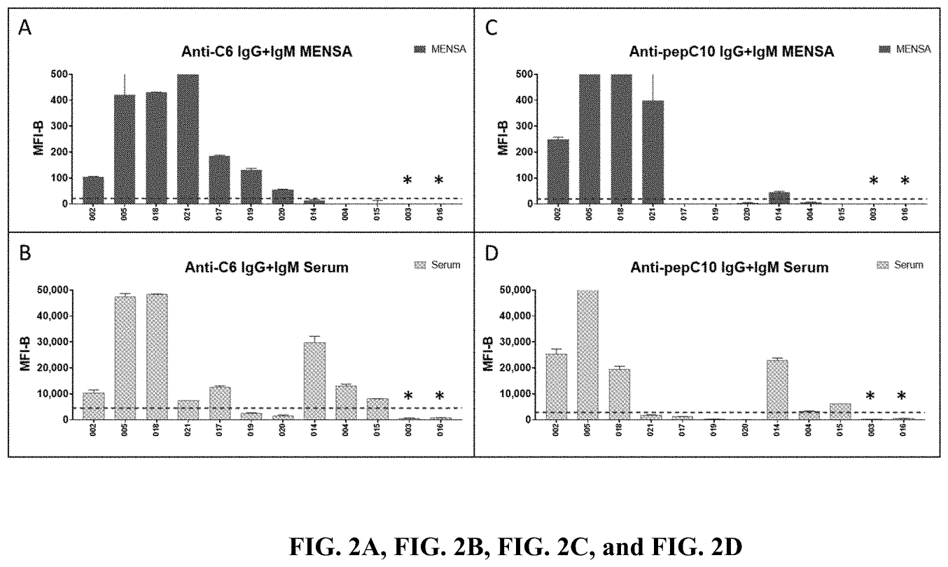

To assess the kinetics of the detectable Lyme disease response in samples from a subject, either Mensa or Serum samples were assayed at both early time points (FIGS. 2A (MENSA) and 2B (serum) and up to 100 days post infection (FIGS. 2C, 2D, 2E, and 2F). FIGS. 2A and 2B show an early positive Lyme C6 response in MENSA prior to a detectable serum response leads to an earlier diagnosis of Lyme disease. FIGS. 2C and 2D show a patient with acute Lyme shows a positive MENSA and serum response early in disease. FIGS. 2C and 2D show the limitations of serum-based detection. Specifically, after successful treatment, MENSA assay shows a rapid decline while serum antibody titers remain positive. FIGS. 2E and 2F show a patient with persistent symptoms including neurologic complications after antibiotic treatment. Although the serum response appears to be declining, it is clear from the MENSA that this patient is demonstrating an ongoing response. Graphs were created from representative patient data for each model. Black dotted line represents origin in MENSA graphs, red dashed line represents the cut-off value for positive in both MENSA and serum graphs: MENSA (22 MFI-B) and serum (4565 MFI-B) respectively. MFI-B: Median Fluorescence Intensity minus Background. Values represent Anti-C6 IgG+IgM.

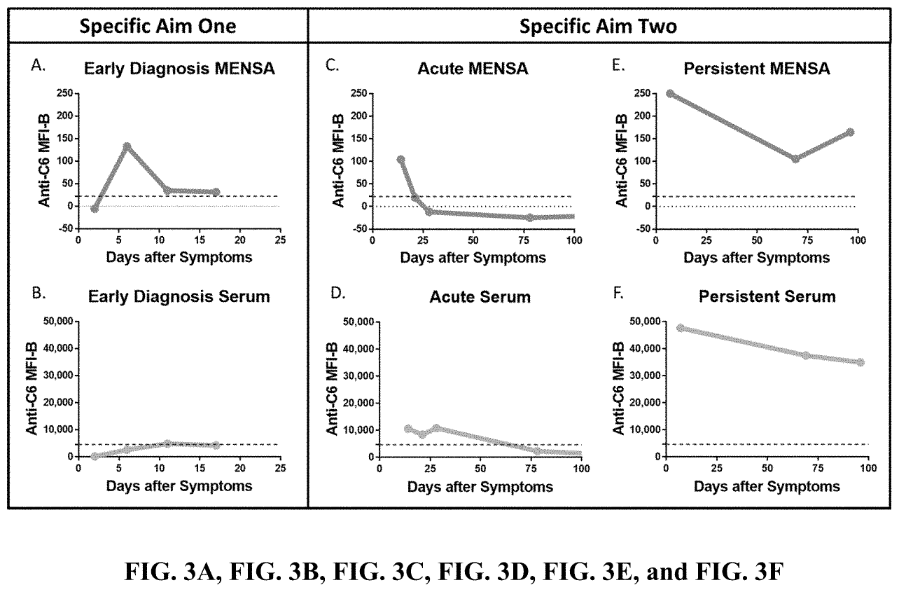

FIG. 3 shows the serum response to 11 Borrelia burgdorferi antigens in two CDC standard sample sets. Seven proteins and two orientations of C6 and pepC10 peptides were coupled to Luminex beads and response was measured against standard serum sample collections from Centers of Disease Control (CDC). Collection sets include serum from Stage 1 Early Lyme patients, Stage 2 Neurologic or Cardiac symptoms of advanced Lyme disease patients, and Stage 3 Lyme arthritis patients. Negative controls include healthy endemic & non-endemic subjects, as well as patients suffering fibromyalgia, rheumatoid arthritis, multiple sclerosis, mononucleosis, syphilis, severe periodontitis. Also tested was TcdB CROP, a non-Lyme marker for C. difficile which appears positive in a small fraction of non-Lyme controls.

SEQUENCES

SEQ ID NO: 1 amino acid sequence of C6 B.
burgdorferi
MKKDDQIAAAIALRGMAKDGKFAVK
SEQ ID NO: 2 amino acid sequence of C6 B.
garinii
MKKDDQIAAAMVLRGMAKDGQFALK

### REFERENCES

Burbelo, P. D., Issa, A. T., Ching, K. H., Cohen, J. I., Iadarola, M. J., & Marques, A. (2010). Rapid, simple, quantitative, and highly sensitive antibody detection for Lyme disease. Clin Vaccine Immunol, 17(6), 904-909. doi:10.1128/CVI.00476-09
Embers, M. E., Liang, F. T., Howell, J. K., Jacobs, M. B., Purcell, J. E., Norris, S. J., . . . Philipp, M. T. (2007). Antigenicity and recombination of VlsE, the antigenic variation protein of Borrelia burgdorferi, in rabbits, a host putatively resistant to long-term infection with this spirochete. FEMS Immunol Med Microbiol, 50(3), 421-429. doi:10.1111/j.1574-695X.2007.00276.x
Liang, F. T., Alvarez, A. L., Gu, Y., Nowling, J. M., Ramamoorthy, R., & Philipp, M. T. (1999). An immunodominant conserved region within the variable domain of VlsE, the variable surface antigen of Borrelia burgdorferi. J Immunol, 163(10), 5566-5573.
Liang, F. T., Steere, A. C., Marques, A. R., Johnson, B. J., Miller, J. N., & Philipp, M. T. (1999). Sensitive and specific serodiagnosis of Lyme disease by enzyme-linked immunosorbent assay with a peptide based on an immunodominant conserved region of Borrelia burgdorferi VlsE. J Clin Microbiol, 37(12), 3990-3996. doi:10.1128/JCM.37.12.3990-3996.1999
Stanek, G., Wormser, G. P., Gray, J., & Strle, F. (2012). Lyme borreliosis. Lancet, 379(9814), 461-473. doi:10.1016/S0140-6736(11)60103-7
Steere, A. C. (2001). Lyme disease. N Engl J Med, 345(2), 115-125. doi:10.1056/NEJM200107123450207

1. A method of distinguishing a new Borrelia Burgdorferi infection from a prior Borrelia Burgdorferi infection in a subject, said method comprising:
a) obtaining a whole blood or a PBMC sample from the subject;
b) separating newly proliferated antibody secreting cells (ASC) from the whole blood or the PBMC sample;
c) generating MENSA by incubating the ASC in a media for supporting antibody production from the ASC; and
d) measuring levels of antibodies specific for antigens of Borrelia Burgdorferi in the MENSA;
wherein the levels of the antibodies specific for the antigens of Borrelia Burgdorferi higher than a control indicates a new Borrelia Burgdorferi infection in the subject.

2. (canceled)

3. (canceled)

4. The method of distinguishing a new Borrelia Burgdorferi infection from a prior Borrelia Burgdorferi infection of claim 1, wherein the newly proliferated ASC are separated from the whole blood or the PBMC sample via magnetic bead pull down or fluorescence acquired cell sorting.

5. The method of distinguishing a new Borrelia Burgdorferi infection from a prior Borrelia Burgdorferi infection of claim 4, wherein magnetic bead pull down or fluorescence acquired cell sorting separates the newly proliferated ASC using antibodies specific for one or more cell surface markers comprising CD38, CD27, CD19, CD138, or IgD.

6. The method of distinguishing a new Borrelia Burgdorferi infection from a prior Borrelia Burgdorferi infection of claim 1, wherein the ASC comprises plasma cells, plasmablasts, or activated B cells.

7. The method of distinguishing a new Borrelia Burgdorferi infection from a prior Borrelia Burgdorferi infection of claim 1, wherein the antibodies are present in MENSA.

8. (canceled)

9. (canceled)

10. The method of distinguishing a new Borrelia Burgdorferi infection from a prior Borrelia Burgdorferi infection of claim 1, wherein the antigen of Borrelia Burgdorferi comprises a whole protein or a fragment thereof.

11. (canceled)

12. A method of diagnosing or detecting the presence of a prior or a new Borrelia Burgdorferi infection in a subject, said method comprising:
a) obtaining a whole blood or a PBMC sample from the subject;
b) separating newly proliferated antibody secreting cells (ASC) from the whole blood or the PBMC sample;
c) generating MENSA by incubating the ASC in a media for supporting antibody production from the ASC; and
d) measuring levels of antibodies specific for antigens of Borrelia Burgdorferi in the MENSA;
wherein the levels of the antibodies specific for the antigens of Borrelia Burgdorferi higher than a control indicates a new Borrelia Burgdorferi infection in the subject.

13. (canceled)

14. (canceled)

15. The method of diagnosing or detecting the presence of a prior or a new Borrelia Burgdorferi infection of claim 12, wherein the newly proliferated ASC are separated from the whole blood or the PBMC sample via magnetic bead pull down or fluorescence acquired cell sorting.

16. The method of diagnosing or detecting the presence of a prior or a new Borrelia Burgdorferi infection of claim 15, wherein magnetic bead pull down or fluorescence acquired cell sorting separates the newly proliferated ASC using antibodies specific for one or more cell surface markers comprising CD38, CD27, CD19, CD138, or IgD.

17. The method of diagnosing or detecting the presence of a prior or a new Borrelia Burgdorferi infection of claim 12, wherein the ASC comprise plasma cells, plasmablasts, or activated B cells.

18. The method of diagnosing or detecting the presence of a prior or a new Borrelia Burgdorferi infection of claim 12, wherein the antibodies are present in MENSA.

19. (canceled)

20. (canceled)

21. The method diagnosing or detecting the presence of a prior or a new Borrelia Burgdorferi infection of claim 12, wherein the antigen of Borrelia Burgdorferi comprises a whole protein or a fragment thereof.

22. (canceled)

23. The method diagnosing or detecting the presence of a prior or a new Borrelia Burgdorferi infection of claim 12, wherein when a new Borrelia Burgdorferi infection is detected, the method further comprises treating the Borrelia Burgdorferi infection by administering to the subject a therapeutically effective amount of a drug for treating Lyme disease.

24. A method of assessing the efficacy of a drug in treating Lyme disease in a subject, said method comprising:
a) obtaining a whole blood or a PBMC sample from the subject around day 3 to day 30 following treatment of the drug;
b) separating an ASC from the whole blood or the PBMC sample;
c) generating MENSA by incubating the ASC in a media for supporting antibody production from the ASC; and
d) measuring levels of antibodies specific for antigens of Borrelia Burgdorferi in the MENSA;
wherein the level of the antibodies specific for the antigens of Borrelia Burgdorferi lower than a control indicates that the drug is effective.

25. (canceled)

26. (canceled)

27. The method of assessing the efficacy of a drug in treating Lyme disease of claim 24, wherein the newly proliferated ASC are separated from the whole blood or the PBMC sample via magnetic bead pull down or fluorescence acquired cell sorting.

28. The method of assessing the efficacy of a drug in treating Lyme disease of claim 27, wherein magnetic bead pull down or fluorescence acquired cell sorting separates the newly proliferated ASC using antibodies specific for one or more cell surface markers comprising CD38, CD27, CD19, CD138, or IgD.

29. The method of assessing the efficacy of a drug in treating Lyme disease of claim 24, wherein the ASC comprises plasma cells, plasmablasts, or activated B cells.

30. The method of assessing the efficacy of a drug in treating Lyme disease of claim 24, wherein the antibodies are present in MENSA.

31. (canceled)

32. (canceled)

33. The method of assessing the efficacy of a drug in treating Lyme disease of claim 24, wherein the antigen of Borrelia Burgdorferi comprises a whole protein or a fragment thereof.

34. (canceled)

35. (canceled)

36. A method of treating, inhibiting, decreasing, reducing, and/or ameliorating a new or ongoing Borrelia Burgdorferi infection in a subject said method comprising
a) obtaining a whole blood or a PBMC sample from the subject;
b) separating newly proliferated antibody secreting cells (ASC) from the whole blood or the PBMC sample;
c) generating MENSA by incubating the ASC in a media for supporting antibody production from the ASC;
d) assaying levels of antibodies specific for antigens of Borrelia Burgdorferi in the MENSA, wherein the levels of the antibodies specific for the antigens of Borrelia Burgdorferi higher than a control indicates a new Borrelia Burgdorferi infection in the subject; and
e) administering a therapeutically effective amount of a drug for treating lyme disease to the subject that assays positive for new or ongoing Borrelia Burgdorferi infection.
